(19) United States
(12) Patent Application Publication
(10) Pub. No.: US 2021/0020060 A1
(43) Pub. Date: Jan. 21, 2021

(54) SYSTEMS AND METHODS FOR SIMULATED REALITY BASED RISK MITIGATION

(71) Applicant: Immersive Health Group, LLC, New York, NY (US)

(72) Inventor: Dov Zacharia Hirsch, Nashville, TN (US)

(21) Appl. No.: 16/933,474

(22) Filed: Jul. 20, 2020

Related U.S. Application Data
(60) Provisional application No. 62/876,414, filed on Jul. 19, 2019.

(51) Int. Cl. G09B 9/00; G06Q 50/26; G06Q 10/06; G06Q 10/10; G06N 5/04; G06N 20/00; G06F 3/01; G08B 21/02

(52) U.S. Cl. CPC G09B 9/00; G06Q 50/265; G06Q 10/0635; G06Q 10/06375; G06Q 10/105; G16H 20/17; G06N 5/04; G06N 20/00; G06F 3/011; G08B 21/02; G06Q 10/06398

(57) ABSTRACT

A simulated reality (SR) generation system is disclosed. The SR generation system includes a processor configured to receive information relating to an entity from one or more data sources, analyze the received information to identify the need of a SR training simulation, and generate the SR training simulation in response to the identification. The SR training simulation is generated by identifying SR simulation components such as a plurality of SR assets, a plurality of interactions between a trainee and SR assets in the SR training simulation, a plurality of interactions between SR assets in the SR training simulation, and/or environmental conditions of the SR training simulation.

### CROSS-REFERENCE TO RELATED APPLICATION

This application claims priority to U.S. Provisional Application No. 62/876,414, filed on Jul. 19, 2019, the disclosure of which is incorporated by reference herein in its entirety.

### TECHNICAL FIELD

The present disclosure relates generally to virtual reality based risk mitigation systems and methods.

### BACKGROUND

A simulated reality (SR) experience provides a three-dimensional (3D) representation of a real or simulated world. Simulated reality encompasses both augmented reality and virtual reality. In an augmented reality experience, a user device receives live image content of a real world environment, and an augmented reality image object is overlaid on the real world environment for display. In a virtual reality experience, a user device receives virtual reality image content of a virtual reality environment, and virtual reality image objects are overlaid on the virtual reality environment for display.

Ever-increasing demands are being placed on entities to assess, monitor, structure, organize, document, track, show, prove, establish or otherwise take action relative to its comprehension, commitment, adherence, compliance, risk mitigation and loss prevention with respect to numerous rules in numerous areas and relating to numerous topics. These tasks require considerable man-power and money expenditure. Moreover, current technology may only permit entities to address disjunctive portions or parts of their needs—for example one of, the development of policies and procedures for risk mitigation and rule compliance, training and education, and assessment or monitoring; but not all.

For example, a simulated reality-based training simulator is a system in which education and training situations in the workplace are implemented using digital content based on real-time simulation, and which is provided with an input/output interface device for allowing a user to directly interact with the content, so that the user can be presented with the same experience that the user would obtain from the actual work environment. When this system is utilized, it is possible for the user to be trained using a procedure that obtains high economic effects, such as the reduction of training-related costs and negligent accidents, and that improves training efficiency. Accordingly, simulation systems corresponding to various situations, such as occur in the space, aeronautical, military, medical, educational and industrial fields, have been developed.

However, the conventional simulated reality-based training simulators have not yet presented various work scenarios that can flexibly cope with all the situations that occur in the workplace. Accordingly, the conventional virtual reality-based training simulators are limited in that they do not fulfill the technical requirements of consumers who desire virtual training-based simulators capable of actively coping with a variety of workplaces and a variety of situations. To date, no accurate, real time model of the complex, constantly changing, interactive relationship between policies, training, and assessment exists, that can be used for building technological products for addressing risk mitigation.

The systems and methods of this disclosure address the issues discussed above.

The information included in this Background section of the specification, including any references cited herein and any description or discussion thereof, is included for technical reference purposes only and is not to be regarded subject matter by which the scope of the invention as defined in the claims is to be bound.

### BRIEF DESCRIPTION OF THE DRAWINGS

FIG. 1 is a diagram of a simulated reality risk mitigation system;

FIG. 2 is a simplified block diagram of a computing device suitable for implantation of the system of FIG. 1;

FIGS. 3A-3C illustrate views of example SR environments at various steps of a SR training simulation; and

FIG. 4 is a flow chart illustrating a method for implementing the virtual reality risk mitigation system of FIG. 1.

### SUMMARY

In some scenarios, disclosed is systems and methods for generating a simulated reality (SR) environment. The system includes a processor and a non-transitory memory containing computer-readable instructions that when executed will cause the processor to perform the methods disclosed herein. The system is configured to receive information relating to an entity from one or more data sources, analyze the received information to identify the need of a SR training simulation, and generate the SR training simulation in response to the identification. The SR training simulation is generated by identifying SR simulation components such as a plurality of SR assets, a plurality of interactions between a trainee and SR assets in the SR training simulation, a plurality of interactions between SR assets in the SR training simulation, and/or environmental conditions of the SR training simulation.

Optionally identifying the need for the SR training simulation may include determining a likelihood of occurrence of a risk event with respect to the entity, and determining that the SR training simulation will reduce the likelihood of occurrence of the risk event.

The system may also be configured to run the SR training simulation on a viewing device suitable for displaying display information in an SR environment based on the SR training simulation.

In some implementations, the system may also be configured to receive a second set of information from the plurality of data source upon completion of the run of the SR training simulation, analyze the received information to determine a performance measure of the entity, and update the SR training simulation based on the analysis.

In various implementations, the environmental conditions of the SR training simulation may be identified based on environmental conditions of a real-world scenario corresponding to the SR training simulation. Optionally, the plurality of interactions between a trainee and SR assets in the SR training simulation may be identified based on, for example, a risk factor for the entity associated with performance of the plurality of interactions, trainee's performance record for performance of the plurality of interactions, and/or trainee's previous training record for performance the plurality of interactions. Similarly, the plurality of assets may be identified based on, for example, a risk factor for the entity associated with an asset, trainee's performance record associated with interactions with an asset, trainee's previous training record associated with interactions with an asset, and/or a real-world scenario associated with the SR training simulation. Examples of assets may include objects, virtual beings, and/or an environment.

In certain implementations, generating the SR training simulation may include identifying the SR simulation components including the plurality of SR assets, and the plurality of interactions between the trainee and SR assets in the SR training simulation.

In certain implementations, generating the SR training simulation may include identifying the SR simulation components including the plurality of SR assets, the plurality of interactions between the trainee and SR assets in the SR training simulation, and environmental conditions of the SR training simulation.

In some scenarios, a risk mitigation simulated reality (SR) systems and methods are disclosed. The system includes a viewing device suitable for displaying display information in an SR environment based on a SR training simulation, an input device configured to allow a user to effect that training simulation, a processor, and a non-transitory memory containing computer-readable instructions that when executed will cause the processor to execute the methods of this disclosure. The system may be configured to receive information relating to an entity from a one or more data sources, analyze the received information to identify a likelihood of occurrence of a risk event with respect to the entity, generate a SR training simulation for reducing the likelihood of occurrence of the risk event, and run the SR training simulation on the viewing device.

Optionally, analyzing the received information to identify the likelihood of occurrence of the risk event with respect to the entity may include analyzing at least one of the following risk factors: a performance score of the entity determined based on past performance of the entity corresponding to the risk event, a learning score of the entity determined based on training history of the entity corresponding to the risk event, various thresholds corresponding to the risk event, a target outcome relating to the risk event, and/or real-time data associated with the risk event.

The system may also be configured to determine whether the identified likelihood of occurrence of the risk event with respect to the entity corresponds to a trigger event. Optionally, the system may generate an alert on a user device in response to determining that the identified likelihood of occurrence of the risk event with respect to the entity corresponds to the trigger event.

In some implementations, system may generate a training regimen based on an analysis of the risk event and the received information, the training regimen configured for reducing the likelihood of occurrence of the risk event. Optionally, the training regimen may include, for example, one or more training modules, frequency of training, required performance measures, training criteria, and/or identification of trainees. The SR training simulation may be generated to run a training module included in the training regimen in simulated reality

The system may cause the system to receive user approval of the training regimen before generation of the SR training simulation.

In certain implementations, generating the SR training simulation may include identifying at least one of the following based on a training module: a plurality of SR assets, a plurality of interactions between a trainee and SR assets in the SR training simulation, a plurality of interactions between SR assets in the SR training simulation, environmental conditions of the SR training simulation, and/or one or more performance metrics of a user participating in the SR training simulation.

The system may also be configured to receive a second set of information from the plurality of data source upon completion of the run of the SR training simulation, analyze the received information to determine a performance measure of the entity with respect to the risk event, and update the SR training simulation based on the analysis.

### SPECIFICATION

In the following detailed description, reference is made to the accompanying drawings, which form a part hereof. In the drawings, similar symbols typically identify similar components, unless context dictates otherwise. The illustrative examples described in the detailed description, drawings, and claims are not meant to be limiting. Other examples may be utilized, and other changes may be made, without departing from the spirit or scope of the subject matter presented herein. It will be readily understood that the aspects of the present disclosure, as generally described herein and illustrated in the Figures, can be arranged, substituted, combined, separated, and designed in a wide variety of different configurations, all of which are implicitly contemplated herein.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. As used in this document, the term “comprising” means “including, but not limited to.” Definitions for additional terms that are relevant to this document are included at the end of this Detailed Description.

The term “entity” can refer to generally herein to individual person, a group of people, or organizations including several groups of people.

Disclosed is a system and method for collecting data in a particular environment, analyzing the data to determine whether a training is needed (e.g., for compliance with certain rules, for risk mitigation, etc.), and deploying a simulated reality training experience in response to determining that training is required. Embodiments of the present disclosure are directed generally to methods for optimizing business processes, complying with governmental regulations, and identifying threat and vulnerability risks for an enterprise. Upon identification of threat and vulnerability risks, the current disclosure also describes embodiments for generating simulated reality based content for mitigating the identified risks.

A simulated reality (SR) experience provides a three-dimensional (3D) representation of a real or simulated world; environments displayed on two-dimensional (2D) devices such as a computer screen, mobile device, or other suitable 2D display; and/or environments displayed in 3D such as on a 3D display or hologram. A SR experience is also referred to herein as a SR environment. Simulated reality encompasses augmented reality, virtual reality, and mixed reality. In an augmented reality experience, a user device receives live image content of a real world environment, and an augmented reality image object is overlaid on the real world environment for display. In a virtual reality experience, a user device receives virtual reality image content of a virtual reality environment, and virtual reality image objects are overlaid on the virtual reality environment for display. Each type of SR system may have objects or assets that are simulations of (i.e., corresponds to) real world items, objects, places, people, or similar entities. The assets or conditions can also provide feedback through haptics, sound or other suitable methods. The SR assets may have positions relative to one another.

Systems and methods disclosed herein suitable for displaying SR environments or experiences. The system and methods also display SR assets relative to specific locations relative to a user's position and movements therein. The assets may comprise media assets, and decorative assets. The media assets could be any of text, image, video, audio, 3D objects or other consumable information-based data. Decorative assets are similar in type to media assets, the difference being that role of the assets in the environments is more cosmetic than content intended to communicate some information and may include, without limitation, simulated walls, furniture, people, or other objects.

A user may interact with an SR environment by controlling the actions of a virtual being (i.e., a participant controlled asset), also known as an “avatar.” The avatar may be a visible and/or audible representation of the user in the simulated environment, and may represent the “body” (in any shape or form) of the user as s/he moves through the simulated environment. Avatars may be able to mimic the actions of real physical people (i.e., users) in a variety of ways, such as by looking in different directions, moving to different locations, entering buildings, handling objects, and even interacting with other avatars in the SR environment. The avatars can include images, audio, and/or biographical information of the participants.

A simulated realty (SR) risk mitigation system as provided herein collects data related to one or more risk factors associated with the operations of an institution and based on those risk factors deploys a risk mitigation SR interface configured to mitigate the actual risk associated with those factors.

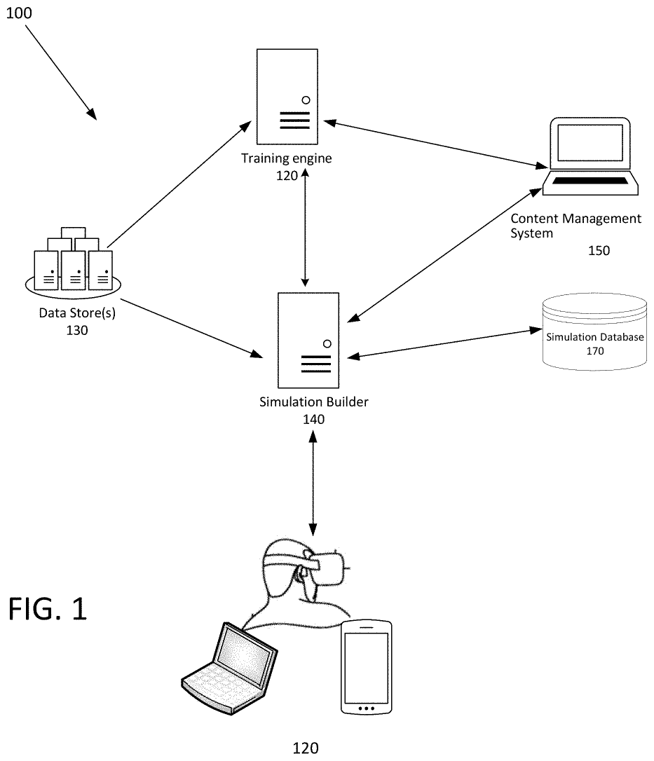

In accordance with various embodiments, as illustrated by way of example in FIG. 1, an SR risk mitigation system 100 includes one or more of a training engine 120, a Simulation builder 140, a content management system 150, a data source 130, and/or an SR interface including an SR device 160. Additional different or fewer components may be provided.

The data store(s) 130 include entity data that is associated with and/or produced by an entity (e.g., a medical facility, a financial institution, an educational institution, etc.). The data store 130 can include a plurality of databases that may relate to an entity's operations and functions. Such data stores may be internal operational databases of an entity (e.g., internal financial records, operations records, personal records, training records, etc.) and/or external databases (e.g., news sources, compliance agency databases, etc.). Optionally, the databases may contain information that the entity considers to be related to entity's risk. For example, a medical facility may rely on databases including electronic medical records (EMR), learning management systems (LMS), charting systems, laboratory results, or ordering systems (labs, medications, etc.; staffing systems and data warehouses), pharmaceutical databases, data records of non-conformance with applicable regulations and policies, potential instances of non-conformance, trends that may result in non-conformance, or the like. In another example, a financial institution may rely on databases including financial transactions. In another example, a university or educational institution may rely on databases including student records or a learning record store (LRS). In another example, a large retail manufacturer or distributor may rely on a database within a supply chain management (SCM) system. It should be noted that while the current disclosure uses a medical facility as an example entity, the systems and methods of the current disclosure may be used in a multitude of different entities and scenarios.

The training engine 120 may receive data from the data sources 130 and perform various analyses to identify instances of risk (“risk events”) to the corresponding entity. Specifically, the training engine 120 is configured to predict the occurrence of a risk event and/or predict the likelihood of occurrence of a risk event. Risk events, as used herein, refer to a negative or unwanted occurrence for an entity. Specifically, the training engine 120 assesses the information obtained from the data stores 130 and scans for relevant patterns and/or a confluence of risk factors indicative of a risk event to an entity.

By predicting risk events, the training engine enables the Simulation builder 140 to generate SR based training(s) to mitigate the occurrence of risk events for the entity. For example, for a medical facility, a risk event may correspond to any scenario that cause financial losses (e.g., higher readmission rates, dissatisfied patients, employee attrition, empty beds, supply wastes, etc.), failure to comply with various rules and regulations (e.g., infections within the facility, unsatisfactory sanitation), legal risks (e.g., litigation risk where a patient is not properly treated or diagnosed because of employee incompetency, etc.), safety risks (e.g., risk of harm to persons and/or property), or the like. Optionally, the system may build a risk profile for an entity and that may be used as a predictive model to determine the total value of risk on a relative risk scale (compared to other entities', risk scale provided by a regulatory agency, etc.), the risk profile being routinely updated.

The training engine 120 may use a predictive model (e.g., a machine learning model, etc.) that utilizes multiple risk factors and/or variables (learned using training data) to predict occurrence of risk events. A predictive model refers to a set of algorithmic routines and parameters that can predict an output(s) of a real-world process (e.g., readmission rates, a diagnosis or treatment of a patient, a suitable recommendation based on a user search query, etc.) based on a set of input features, without being explicitly programmed. A structure of the software routines (e.g., number of subroutines and relation between them) and/or the values of the parameters can be determined in a training process, which can use actual results of the real-world process that is being modeled. Such systems or models are understood to be necessarily rooted in computer technology, and in fact, cannot be implemented or even exist in the absence of computing technology. While machine learning systems utilize various types of statistical analyses, machine learning systems are distinguished from statistical analyses by virtue of the ability to learn without explicit programming and being rooted in computer technology. In some embodiments, the machine learning system may use a classifier that typically starts with a set of labeled or unlabeled training data and applies one or more algorithms to detect one or more features and/or patterns within data that correspond to various labels or classes (e.g., occurrence of a risk event probabilities). The algorithms may include, without limitation, those as simple as decision trees, as complex as Naïve Bayes classification, and/or intermediate algorithms such as k-nearest neighbor. Classifiers may include artificial neural networks (ANNs), support vector machine classifiers, and/or any of a host of different types of classifiers. Once trained, the classifier may then classify new data points using the knowledge base that it learned during training. The process of training a classifier can evolve over time, as classifiers may be periodically trained on updated data, and they may learn from being provided information about data that they may have misclassified.

The system may also use a statistical model that is configured to find relationships between risk factors to predict the occurrence and/or likelihood of occurrence of a risk event.

Alternatively and/or additionally, the training engine 120 may use a pre-defined rule set (e.g., a look up table) that includes various risk factors and corresponding mapping with one or more risk events.

The number of risk factors utilized can be extensive and include, without limitation, a performance score of an entity relating to a risk event (i.e., how likely is the occurrence of a risk event given the entity's part history), a learning score of an entity relating to a risk event (i.e., training history of the medical facility population for mitigating the occurrence or outcome of the risk event), real-time data (e.g., real-time data indicative of current events, trends, future events, or the like), various thresholds, entity specific outcome targets, or the like.

The performance score (or a rating indicative of a comparative level such as good, bad, satisfactory) may be a score that quantifies the performance of an entity given its historical performance and may be determined based on a rule set (e.g., based on one or more factors such as patient provider ratios, admission diagnosis, patient profile etc.), a predictive model (e.g., a machine learning model trained on past readmissions and associated factors), or the like. For example, if the risk event is readmission rates in a medical facility becoming greater than a threshold value, the performance score of the medical facility for the readmission rates may be a likelihood that is assigned to each new admission that there will be a readmission of the same patient within a certain time period (e.g., 30 days) given the patient profile, type of ailment, severity of ailment, environmental factors, etc.

The learning score may be determined to assign a baseline score (or a rating indicative of a comparative level such as good, bad, satisfactory) to an entity's current learning and/or training status. The learning score may be determined using, for example, a statistical model, a machine learning model, a rule set, etc. that take into account factors such as training objectives for the entity (at a group level or individual level within the entity), training infrastructure (e.g., budget, technology, etc.), rate of training (e.g., volume, frequency, progression, completion, etc.), training prioritization (e.g., based on compliance requirements, licensure requirements, career upskilling, etc.), past training data, past training evaluations, or the like. For example, if the risk event is readmission rates in a medical facility becoming greater than a threshold value, the learning score may be a score that quantifies an entity's current training status for reducing the readmission rates. In this example, if readmission rates are reduced by properly training a patient in the performance of a particular breathing exercise before discharge, information relating to the training of the hospital personnel (e.g., quality, frequency, expiration, etc.) in providing such training to the patients may be a factor (in addition to other factors) may be used in determining the learning score of the entity and/or an individual personnel. Optionally, a learning score may be determined for individuals of an entity.

Thresholds refer to numerical values that may be used for comparison for, for example, determining that a risk event will occur, a risk event is likely to occur, determine that some action must be taken, identifying triggers relating to various likelihood of occurrences of a risk event (e.g., readmission rates going over 10%, 20%, 30%, or the like). Outcome targets may be a desired likelihood of a risk even occurrence, and may be provided by an entity (e.g., a medical facility may want to keep the readmission rates below 15%, below a previous year's readmission rate, below a national average readmission rate, etc.).

In some embodiments, the predictive model utilizes goal oriented machine learning to identify and quantify risk events (or levels) based on relationships between the risk factors (each risk factor may be assigned different weights). Optionally, the predictive model may be trained specifically for each entity in order to produce more accurate predictions for the relevant populations of the entity (e.g., patient and provider populations of a medical facility).

For example, criteria may be defined based on the applicable regulations and policies, and thresholds established. When the monitored data exceeds certain thresholds, that may trigger an indication of actual or potential non-compliance of applicable regulations or policies. Rules may be defined based on applicable regulations and policies, and then applied to the collected compliance data to identify actual or potential occurrences of non-compliance, and so on.

As discussed above, the training engine 120 may, optionally, use a rule set including a mapping (and/or weights) of risk factors to risk events for predicting occurrence of a risk event and/or likelihood of occurrence of a risk event. For example, for a risk event in a medical facility corresponding to readmission rates (i.e., readmission of a patient within a certain time of discharge such as within 30 days), the system may include a rule set that defines various weights for one or more risk factors and a mapping to readmission rates. Examples of risk factors can include training history of employees relating to protocols for reducing readmission rates (based on, for example, known best practices), past readmission rates in different months, current infections prevalent in the geographical area that are known to cause a spike in readmission of certain patients (for e.g., flu may be known to cause readmission of patients over 65 years of ages), diagnosis of currently admitted patients (for example, pulmonary related admissions may be known to be more likely to be readmitted within a certain time period). Each of these risk factors may be assigned a certain weight and may be combined according to the mapping (e.g., added, averaged, etc.) to determine the likelihood of readmission rates going up or down.

For example, the training engine 120 may determine that rates of readmissions are likely to increase over the next 30 days (using a predictive model or a rule set) upon detection of the following risk factors (based on the data from the data stores 130): (i) there is a spike in current pulmonary related admissions (based on EMR data), (ii) the Center for Disease Control (CDC) has issued a flu alert (from the CDC website), (iii) the current performance score of the medical facility for readmissions relating to pulmonary related admission is poor, and/or (iv) the learning score relating to training provided for reducing readmission rates overall and/or relating to pulmonary admissions is satisfactory (or poor because training frequency is not satisfactory, or past trainings are about to expire).

Optionally, the training engine 120 may also determine that the nature of risk event requires generation of an alert and/or a training regimen. For example, if the current readmission rate is 13% and/or the target readmission rate of for the year is 15%, such that the predicted rise in readmissions rate will lead to the entity not meeting its target, the training engine 120 may generate an alert and/or a training regimen. In another example, if the rate of readmission is likely to increase over a threshold, the training engine 120 may generate an alert and/or a training regimen.

An alert can be distributed as a suitable message to a user (via, for example, a user device, an audio interface, etc.) such as a training administrator and/or other personnel of the entity. For example, the alert may be notifications that can include text message (SMS), email, automated phone call, or other suitable communication method. The notification informs the administrator that a specific risk event is going to occur and/or is at heightened likelihood of occurring. The messages can also be tailored to the requirements of the entity. For example, a health institution may limit the message to be HIPAA compliant. The notification may also include or provide a link or reference to a proposed training regimen.

The training engine 120 may also determine the nature of the risk event and generate a proposed training regimen to mitigate the risk (i.e., prevent the risk event from occurring and/or reduce the likelihood of its occurrence below an acceptable level). A training regimen may include one or more SR-based training modules for various entity personnel, frequency of training, required performance metrics, training criteria, identification of trainees, or the like. A training module may be a data file (or a collection of data files) that describe a training scenario for a trainee and may include a plurality of training related steps to be performed by a trainee.

As discussed above, in some embodiments, the training engine 120 may generate the proposed training regimen in response to a triggering event (e.g., when a specific threshold is met with respect to the risk event). A trigger may be a condition that causes the training engine 120 to propose a training regimen. It can be based on measuring data and comparing them to a threshold. For e.g. a value above or below a threshold would cause a trigger. In another example, occurrence of an event such as an upcoming compliance check would cause a trigger. It should be noted that the training regimen and/or the simulation may be generated in response to a user instruction even in the absence of a trigger.

For example, for a risk event in a medical facility corresponding to readmission rates, the training engine 120 may generate the training regimen if it determines (based on the risk factors discussed above) that the readmission rates are likely to rise above 15%. The thresholds can be unique to each entity, and can be set automatically by the training engine 120 based on predetermined performance targets, and/or manually determined or adjusted. In the above example, the threshold may be automatically determined based on, for example, a target outcome of keeping the annual readmission less than the previous year's readmission rate (e.g., 20%), time remaining in the year, predicted amount of increase in the readmission rate, types/frequency of training that will be required to keep the readmission rates at the desired level, or the like.

For generating the proposed training regimen, the training engine may use a predictive model (e.g., a machine learning model) that is configured (e.g., using training data) to identify various training areas, subjects, scenarios, etc. in which providing timely training to the entity personnel will either prevent the risk event from occurring and/or reduce the likelihood of its occurrence below an acceptable level. Training engine 120 may then use the predictive model to generate a training regimen that the predictive model has learned will fill any known gaps in the identified areas, subjects, scenarios, etc. Such area, subjects, scenarios, etc. as well as the training regimen may be determined by providing as input, for example, the risk factors identified that led to a detection of the risk event, the entity's performance and learning scores, real-time conditions and data, the threshold, the target outcome to a previously trained predictive model.

For example, if the training engine determines that the rate of readmissions is likely to increase over a threshold such that the medical facilities' target outcome may not be achieved, it may use a predictive model that correlates the current performance score (e.g., poor readmission rates for patients relating to pulmonary-related diagnosis), current learning score (e.g., previous trainings completed, performance on previous trainings, expiry dates of previous trainings, etc.), current rate of readmissions (e.g., 13%) compared to a target goal (e.g., 15%), predicted increase in the rate of readmissions, etc., real-time data (e.g., flu season, diagnosis of current admitted patients, etc.) with training areas that the predictive model has learned are likely to bring down the readmission rate for that entity, and generate a training regimen accordingly. In this example, if the training engine 120 has determined that the readmissions rates are likely to increase because: (i) there is a spike in current pulmonary related admissions (based on EMR data), (ii) the Center for Disease Control (CDC) has issued a flu alert (from the CDC website), (iii) the current performance score of the medical facility for readmissions relating to pulmonary related admission is poor, and/or (iv) the learning score relating to training provided for reducing readmission rates overall and/or relating to pulmonary admissions is satisfactory (or poor because training frequency is not satisfactory, or past trainings are about to expire), the training engine 120 may identify training areas relating to proper discharge of patients that the predictive model has learned may reduce readmission rates. For example, the predictive model may identify that properly instructing patients regarding, for example and without limitation, smoking cessation, breathing exercises (e.g., pursed lip inhalation), use of medical devices (e.g., inhalers, masks, etc.), dose and usage of prescribed medication, medication refills, sanitation procedures, and providing related documents before discharge may reduce the likelihood of readmission of such pulmonary related patients.

The predictive model may then generate a training regimen for training of personnel involved in patient discharge based on the performance score, learning score, baseline profile, etc. of the entity. It should be noted that the training regimen may be individually tailored to different personnel, groups of personnel, etc. in the entity based on their unique circumstances (e.g., previous trainings completed, past performance data, role within the entity with respect to the risk event, or the like).For example, the training engine may determine that nursing personnel are involved in instructing patients in the proper use of medical devices such as inhalers, and were provided training for instructing patients at a time that doesn't satisfy the requirements reducing readmission rates (or their performance in previous training was unsatisfactory), and/or their current performance is unsatisfactory (determined based on documentations, patient reviews etc.), and may generate a training regimen that requires such nursing personnel to undergo training in giving proper instructions to patients at discharge time regarding the use of inhalers. The training regimen may further be personalized on an individual basis such as, for example, if a training module for providing proper instruction to a patient in the use of inhalers requires each nursing personnel to go through 5 instructional steps (discussed below in more detail), the training module for each personnel may focus on the steps for which a personnel is known to have received improper training or exhibit poor performance in (for example, make the step relating to selection of a proper inhaler harder by giving more choices) Similarly, the frequency of training, identification of trainee, performance metrics, feedback, etc. in a training regimen may be individually tailored.

Additionally and/or alternatively, the training engine may use a rule set for generating the proposed training regimen. The rule set may include a mapping between the risk event (and/or risk factors, likelihood of risk event, etc.) and, to identify various training areas, subjects, scenarios, etc. in which providing timely training to the entity personnel will either prevent the risk event from occurring and/or reduce the likelihood of its occurrence below an acceptable level for that entity. The rule set may be provided based on, for example, known best practices or factors for prevent the risk event from occurring and/or reduce the likelihood of its occurrence below an acceptable level for that entity. The rule set may also include instructions for generating a training regimen that is known to fill gaps in the identified areas, subjects, scenarios, etc. For example, a training module can be identified based on saved scenarios suitable to address the particular risk event at issue.

For example, the rule set may include a mapping between lowering the readmissions rates for pulmonary patients and proper instructions to patients at discharge regarding, for example and without limitation, smoking cessation, breathing exercises (e.g., pursed lip inhalation), use of medical devices (e.g., inhalers, masks, etc.), dose and usage of prescribed medication, medication refills, sanitation procedures, and providing related document. The training engine may use the rule set and data relating to current training or performance status of entity or entity personnel to identify areas where additional training may be required. The training engine may then generate training modules that are designed to train personnel in giving proper instructions to patients at discharge time in the identified areas. For example, the training module may be generated to include one or more steps to be performed while instructing the patients, the steps being determined based on known best practices for providing instructions (e.g., in breathing exercises, use of inhalers, etc.). Optionally, the rule set may include a mapping between lowering the readmissions rates for pulmonary patients and various previously stored training modules. The training engine may use the rule set and data relating to current training or performance status of entity or entity personnel to identify the appropriate training modules.

The training module include in the training regimen can be presented as a recommended SR simulation suitable to convey to a user the information or training suitable to mitigate the particular risk event that triggered the system. As such, a training module of a training regimen can include the selection or generation of a proposed SR simulation, a type of SR simulation, or a proposed list of criteria to utilize in an SR simulation.

Referring back to FIG. 1, the training engine 120 may transmit the proposed training criteria to a simulation builder 140. It should be noted that while the simulation builder 140 and training engine 120 are illustrated as separate components in FIG. 1, they may be a single component. Moreover, some or all of the functions of the training engine 120 may be performed by the simulation builder 140, or vice versa. The simulation builder 140 is configured to create a SR simulation environment (also referred to as a training simulation) including assets and conditions for interactions with those assets in order to implement the training modules included in the training regimen in SR, upon receipt of a training regimen from the training engine 120. Optionally, the simulation builder 140 may be configured to generate the simulation environment in response to a triggering event (e.g., detection of a risk event, identification of risk factors, etc.) without reliance on a training regimen. Specifically, the simulation builder 140 may build the simulation directly based on one or more conditions associated with the risk event, previously stored rules relating to simulation building, training gaps identified, or the like. A trigger is a condition that causes the simulation engine 140 to start the simulation building process. It can be based on measuring data and comparing them to a threshold. For example, a value above or below a threshold would cause a trigger. Similarly, a trigger may be an identification of a training gap for an entity personnel (e.g., previously performed training is about to expire, previously performed training needs to be updated, to address performance issues, etc.). The simulation builder 140 may be configured to generate the simulation environment in response to a user request (e.g., an administrator may determine that a particular employee need training to improve hand washing skills in light of recently updated protocols and may request the simulation builder to build a SR simulation for the training based on the new protocol).

A simulated scene may include SR components such as assets, conditions for interactions with and/or between assets, various environmental conditions (e.g., amount of light, wind, etc.). An asset can be any suitable object that the user should interact with in order to reap the benefit of the proposed training. Categories of assets that may be used to address risk include, but are not limited to the following: Objects (i.e. furniture, doors, tools, instruments, vehicles, electronic equipment, machines, computers, buildings, natural elements such as water); Places/Environments (i.e. bedroom, garden, factory, buildings, outer space, other planets); Virtual Beings (i.e. people or animals); etc. Interactions with assets include manipulation, contact, and/or other types of interactions between a real-world user (directly and/or via an avatar) with an SR asset and/or between SR assets (e.g., picking something up, bodily motion, engaging with another person, operating a machine, applying force to an object, manipulating an object, voice recognition). Such interactions may be built into the simulated scenario by for example, configuring an asset to react based on a detected gesture of a user (e.g., touch, push, pull, etc.) Types of assets, interactions, and environments can be stored in other systems or databases and may be received by the simulation generator 140. For example, these assets, interactions, environments, etc. can be hosted and stored on a secure server. Assets as well as interactions may be identified based on the training module. For example, in a healthcare institution, the object asset could be a syringe with the expected interaction of preparing and administering the syringe correctly. Similarly, the assets could include an object asset inhaler and a virtual being (patient) for training a person in providing proper instructions to a patient regarding the use of the inhaler. In another example, in the manufacturing industry, an object asset could be a type of tool used to fix a machine. Information relating to interactions can be determined from the received training module and the known physics (or physical properties) of various objects. For example, if the training module relates to providing proper instructions to a patient regarding the use of the inhaler, the interactions may be determined based on the steps required to be included in the training module, such as picking up of inhaler asset, assembling the inhaler, interaction with a patient chart asset, interaction with a patient asset, etc.). Similarly, environmental conditions of a simulation may be generated based on the received training module, corresponding real-world scenarios, or the like.

Specifically, the simulation builder 140 may identify various SR components and use the components to generate the SR simulation for a training module based on the training steps included in the training module, the performance metrics associated with the training module, the required interactions in the training module, the real-world scenario corresponding to the training module (e.g., location, required assets, environment, etc.), user instructions, trainee and/or entity characteristics, or the like. For example, the simulation module may select different types of assets for generating the training module based on the above factors. The simulation builder 140 may also receive information from information stores such as, weather patterns, device profiles and physics, photographs relating to environments, transportation patterns, or other information suitable for building SR simulations.

Using the above example training module of training medical personnel in properly providing instructions to a patient at the time discharge, particularly in the use of inhalers, the simulation builder 140 may first assess the requirements of the training module to generate the simulation. For example, the training module may require the trainee to perform the following steps, and evaluation of the trainee based on performance of each step: (i) review patient chart and confirm that the patient in the room is the patient being discharged; (ii) accurately select an inhaler based on the patient chart information; (iii) demonstrate to the patient how to properly assemble the inhaler (e.g., include a spacer if required); (iv) demonstrate to the patient to properly press the inhaler plunger and keep it pressed for at least 30 seconds (for proper vaporization of medicine, may also require the trainee to demonstrate checking of vapor formation); (v) demonstrate to the patient to timely release the plunger and inhale deeply; (vii) demonstrate how a correctly metered dose of medication when properly vaporized affects the Lungs (compared to incorrect dose or vaporization); (vii) put the inhaler down and select a new inhaler to give to the patient; (viii) review the patient's use of the new inhaler and/or provide feedback to patient on the use and/or answer questions (based on voice recognition); and (ix) document the training to receive patient's acknowledgement. The training module may also require the personnel the complete the training in a certain time window (e.g., 15 minutes).

Upon review of the training module, the simulation builder 140 may first select an environment for simulating the training module. An environment may be determined based on the real-world scenario or location corresponding to the training module. In the current example, a medical facility personnel (in a real world) will typically instruct a patient in the proper use of the inhaler in, for example, a discharge room, an exam room, a nursing station, outpatient room, etc. of a medical facility. As such, the simulation builder 140 may create an environment that is similar to the real-world scenario corresponding to a location where such instructions will be provided (e.g., exam room). The simulation builder 140 may similarly select assets such as furniture, hand washing areas, a towel dispenser, a stethoscope, a blood pressure monitor, a patient chart, a desk, etc., that are typically included in an exam room. Optionally, the simulation builder 140 may have access to photographs of actual exam rooms in the particular entity and create an environment that is similar to such exam rooms.

Next the simulation builder 140 may select assets particularly required for the training module. For example, in the training module of training medical personnel in properly providing instructions to a patient at the time discharge, the simulation builder 140 may determine that at least the following object assets will be required: a plurality of different types of inhalers (e.g., arranged on a shelf); a wheelchair for the virtual patient to sit on, a patient chart (e.g., a clipboard with a chart or an electronic device showing the chart), written material to give to a patient, an inhaler that corresponds to the right selection of the inhaler for a given training scenario (e.g., training scenario requiring the use of inhaler type A from amongst inhaler types A, B, C, and D), a 3D set of lungs to demonstrate how the medication affects the lungs, etc. The simulation builder 140 may choose inhalers A, B, C, and D based on a personnel's previous performance history (e.g., if an individual is known to select inhaler B instead of A for a particular use case the simulation builder may include inhalers A and B in the simulation for proper training), similarity between the inhalers, etc. The simulation builder 140 may also create or generate a virtual patient based on, for example, a patient profile that corresponds to the risk event of high readmission rates. For example, if patients between the age of 55-65 and of a particular race/ethnicity are known to have a higher rate of readmissions for the entity after a first admission relating to a pulmonary condition (i.e., are related to a risk event), the system may create the virtual patient to be in that age group and that particular race/ethnicity. If the gender of the patient is not related to risk event, the simulation builder may randomly assign a gender to the patient. As such, some of the assets (objects, interactions, environment, and/or virtual beings) may be randomly generated, while others may be customized based on the risk event identified, risk factors, the generated training regimen, a particular training module, or other information. Optionally, some characteristics of the assets may be randomly selected while others are customized.

The generated simulation may also include instructions for the trainee in the form of, for example, text, audio and/or video instructions, symbols (e.g., arrows pointing to assets that should be used next), clock timers (e.g., to show time for pressing a plunger), or the like. Optionally, such instructions may be overlaid on the simulation environment. Such instructions may be provided to the trainee at the beginning, during the training run, upon performance of a wrong step, incorrect performance of a step, at the end, or the like.

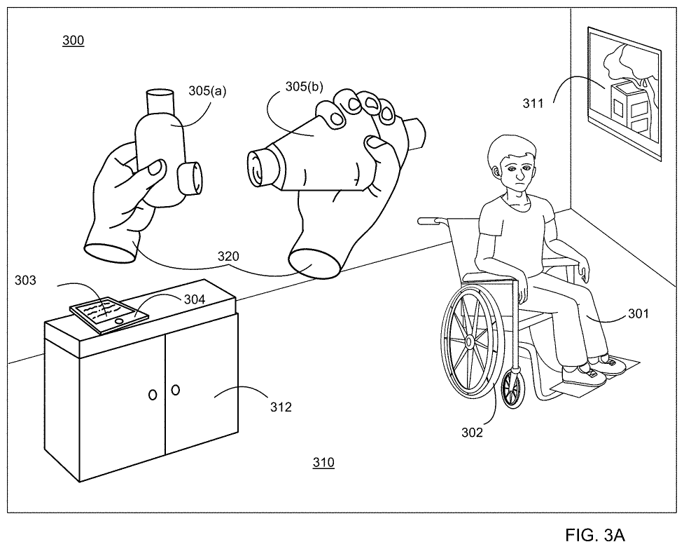

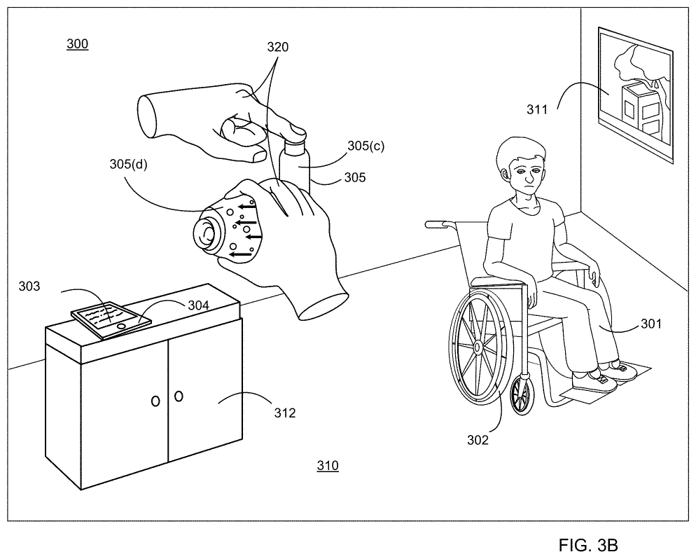

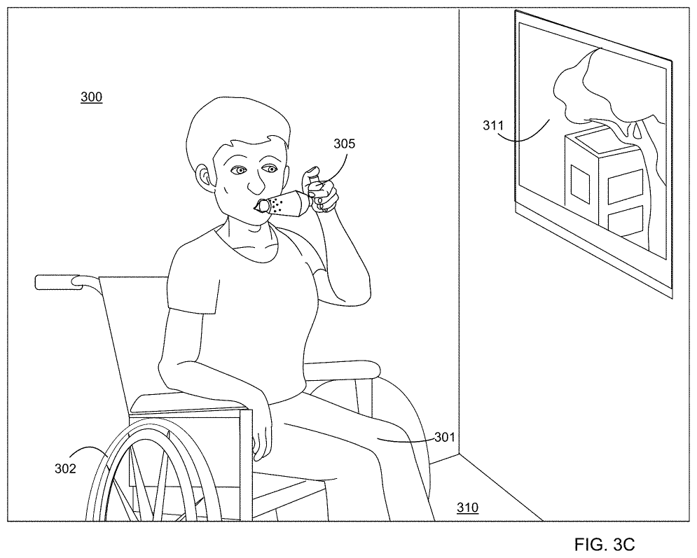

An example simulated environment 300 corresponding to different steps of the training module for training medical personnel in properly providing instructions to a patient at the time discharge is shown in FIGS. 3A-3C. FIG. 3A is a simulation of a trainee demonstrating the assembly of inhaler parts, FIG. 3B is a simulation of a trainee demonstrating proper charging the inhaler by pressing down till medicine vaporize, and FIG. 3C is a simulation of a trainee reviewing patient's use of the inhaler in order to provide feedback, respectively. As shown in FIG. 3A, the generated simulated environment 300 includes an exam room 310 having a window 311 and a desk 312. The simulated environment 300 also includes assets that a potential trainee will interact with such as a virtual patient 301 (sitting in a wheelchair 302), a patient chart 303 (displayed on an electronic device 304), and parts of an inhaler device 305 (nebulizer 305(a) and spacer 305(b)). Finally, the generated simulated environment 300 may include a simulation of the trainee (e.g., an avatar). In the examples shown in FIGS. 3A-3C, the avatar only includes hands 320 (however, a full-body or other partial avatars are within the scope of this disclosure). While not shown here, the simulated environment 300 may also include, without limitation, a wash area, a towel dispenser, stethoscope, written material to give to the patient, a selection of inhalers, etc. during one or more steps of the training.

The simulation builder 140 may also analyze the training module to generate various interactions within the simulated environment. For example, in the above simulated environment 300, the trainee (i.e., via hands 320) will need to interact with the patient chart 303 to, for example, verify patient identity, analyze patient history to select the right type of inhaler, document the training, receive patient acknowledgement, etc. The simulation builder 140 may, therefore, configure the patient chart such that picking up of the patient chart and editing of the patient chart can be simulated for the benefit of the trainee in response to detecting corresponding actions (e.g., picking up action and force, writing using an instrument, etc.). Similarly, the trainee will need to interact with the nebulizer 305(a) and spacer 305(b) to demonstrate their properly assembly, and to demonstrate the use of the assembled inhaler 305. The simulation builder 140 may, therefore, configure the inhaler parts such that they are displayed as being joined to each other if assembled properly by the trainee (or fall apart if not assembled properly) (as shown in FIG. 3B and FIG. 3C). Similarly, the simulation builder 140 may configure the assembled inhaler 305 to include a plunger 305(c) that can be pressed down in response to detecting a downwards force from the trainee at the plunger 305(c) Similarly, the simulation builder 140 may configure the assembled inhaler 305 to simulate formation of vaporized medicine 305(d) upon detection of the plunger 305(c) being pressed for at least 30 second (and no vaporization before 30 seconds).

The simulation builder 140 may also analyze the training module to identify one or more performance measure associated with one or more steps included in the training module, and configure the generated simulation for analyzing the performance measures. In the above example, performance measures can include, for example, time taken to complete the training, correctly following the order of the steps, use of correct equipment (e.g., correct identification of the inhaler); responding to patient questions accurately; analyzing and correcting the patient's use of the inhaler; pressing the plunger for at least 30 sec; correctly measuring the medicine dose; correctly holding the inhaler, etc. Therefore, as discussed above, the generated simulation may include a variety of inhalers from which the trainee must select the correct type. The variety may be configured to make it harder or easier for the trainee to make the selection (e.g., include inhalers that look similar to make the selection harder, include inhalers that are known to be incorrectly picked for a simulated condition, etc.). Additionally, the inhaler may be programmed to show vaporization only when the plunger is pressed for 30 seconds Similarly, the virtual patient may be programmed to perform one or more steps of using the inhaler incorrectly to solicit a corrective feedback action from the trainee. In another example, the virtual patient may be programmed to ask questions during a demonstration to get the trainee to provide an answer.

Once the simulation is generated, the simulation is delivered to a SR device 160 and the training simulation is run. The SR device can be any suitable viewing device configured for the type of SR environment that is meant to run on the device. For example, the device can be a head mounted display (HMD) configured to run a virtual reality environment. As discussed above, other environments can run on other platforms as well. For example, the device can be a handheld device operating an augmented reality system. Alternatively, the device can be a tablet or a PC providing a flat display of a three dimensional environment; a suitable viewable surface, such as a television or monitor; a wearable device operating an augmented reality system, such as a smart watch or smart glasses; an implanted device, or other suitable devices or systems.

A system for displaying a simulated reality environment is provided herein. In accordance with various embodiments discussed herein, the system allows a trainee to manipulate perspectives of his/her avatar to interact with the SR assets. An example of one such user interaction could be a user represented as hands in FIGS. 3A-3C handling the inhaler.

In accordance with various embodiments, the systems disclosed herein allow a user to run and participate in simulated trainings, using an application on an immersive or non-immersive devices. In accordance with various embodiments, immersive devices can emulate a user's head, body, and/or hand position. The position of the head, body, and/or hands can be represented in the simulated reality environment such that other users see the virtual position of the emulated user position. In some embodiments, the position of the non-immersive device can also manipulate the perspective by which the user views the virtual reality environment via the non-immersive device and/or other users see the virtual position of a user associated with the non-immersive device in the virtual reality environment. Such manipulation can be accomplished when using movable devices, such as smart phones, tablets, and augmented reality glasses. In such embodiments, a user can move their device around them, looking in any direction, and see on their device's screen, that portion of the virtual reality environment that would be seen were the user in the virtual reality system looking in that direction. The correct position, orientation, and point of view of the user may be determined by input from the devices sensors (described below). In accordance with various embodiments, to the users using the system from immersive devices such as headsets, any non-immersive user appears as a participant controlled asset (e.g. avatar) with that participant controlled asset having position, orientation, and movement within the environment. The position of the head and/or hands of the participant controlled assets may be positioned relative to where the user is holding the device. In some embodiments, additional adjustments to the head and/or hands may be made via user input elements and/or input devices.

Once the user has completed the simulation, the users performance is measured. The performance measurement can be a rating of the performance or competency of the user's interactions in the simulation at various steps. For example, the system may rate or score the performance based on completion of the training in a certain time period, performing the training steps in the correct order, completion of each step of the training, correct selection of the inhaler, proper demo of the inhaler assembly and usage, correct metered dosage, proper response to patient questions, or the like. Optionally, each mistake by the trainee may result in lowering of the score, while each correct step may result in an increase in the trainee's score. Furthermore, different performance measures may be assigned different weights in determining the overall performance. For example, failure to identify the correct inhaler may result in failing the training (irrespective of results of other performance measures), and the simulation may prompt the trainees to restart the training. Such performance measures and associated actions may be configured into the interactions of the simulated training.

Optionally, performance and competency data generated during training and/or data collected during real-world scenarios for which the training was provided can be used to evaluate whether or not the simulation needs to be updated (e.g., if the simulation did address the gaps in training and/or did not improve performance) and/or if the simulation was suitable to prevent occurrence of the risk even/reduce the likelihood of occurrence of the risk event. If the simulation is determined to be unsuitable to prevent occurrence of the risk even/reduce the likelihood of occurrence of the risk event (i.e., performance of trained personnel during and/or after training does not meet a threshold) then the simulation may be rejected and the system may send the information back to the training engine 120 to propose a new simulation. This feedback loop allows for future improved performance of the system 100. If the performance of trained personnel during and/or after training is acceptable, the system may close the process and may await the next risk trigger. An artificial intelligence (AI) builder or machine learning (ML) engine can be implemented to adjust and maximize the improvements in performance of the proposed simulations to achieve better outcomes and limit adverse events.

It should be noted that while the above disclosure describes detection of a single risk event and subsequent generation of a training regimen and/or a training simulation, the disclosure is not so limiting. Specifically, the system may determine that more than one risk events are likely to occur simultaneously, and generate training regimen and/or a training simulation directed to reduce the likelihood of occurrence of each risk event individually or as one or more groups of risk events (having common risk factors). Determination of occurrence of risk events may be correlated to each other (e.g., increase in likelihood of occurrence of one risk event may be used as a risk factor for determining the likelihood of occurrence of another risk event). For example, to addition to readmission rates, the system may also determine, based on one or more risk factors, a likelihood of shortage of ventilators (e.g., high pulmonary related admissions, flu season, etc.), in addition to prediction of a likelihood of higher readmission rates. As such, a training regimen designed to decrease the likelihood of occurrence of high readmission rates will also impact the risk event relating to shortage of ventilators. Optionally, the risk events may be independent of each other and may require different training regimens.

Referring back to FIG. 1, the system 100 may also include a content management system 150. This content management system 150 may be a local application on a user device (e.g. phone, tablet, PC, HMD, etc.). Additionally and/or alternately, the management system 150 is hosted on a different device and is delivered as part of network or web based content to the user.

The user may be, for example, a training administrator of the entity. The proposed training regimen and/or the generated simulation may be presented to the user, via the content management system 150, for approval, modification, and/or rejection. For example, the training regimen may be presented to an administrator for approval before generation of the simulation by the simulation generator 140. If the proposal is accepted, the system moves on to generating the SR simulation via the simulation builder 140. If the proposal is modified, the user provides feedback to the system to include additional or fewer criteria or to change the parameters of the risk mitigation criteria provided. For example, the administrator may choose to modify the competencies, steps/tasks involved, the sequence involved, the types of virtual humans (i.e. swap an adult for a child), the virtual human's demeanor (irate to calm), the environment (change from a public setting, such as a restaurant, to a private setting, such as a home), add a synchronous (i.e. multi-user) capability for group training, etc. The training engine 120 can then build a new proposal that is reevaluated by the administrator. Alternatively, the new proposal based on the administrator's input can be sent directly to the simulation builder 140. The administrator can also reject the proposal. For example, a rejected and/or modified proposal informs the training engine (120) that it did not correctly identify a necessity to recommend a SR training based on the data sources. The user may reject the proposal without cause, or specify how the recommended SR training was misaligned with the perceived risk. The training engine 120 then uses the information provided by the administrator to “learn” why its recommendation was incorrect, informing a greater degree of accuracy for future possible recommendations. After ingesting the new information the system documents receipt and recalculation of the risk that initiated the recommended SR training in a searchable database to begin with.

Similarly, the generated simulation can also be sent to the user for approval, modification, and/or rejection. The simulation generator 140 may learn from user's actions. For example, a rejected and/or modified simulation informs the simulation generator 140 that it did not correctly identify assets, instructions, measurement criteria, etc.; and may use it for future simulation generations.

Referring back to FIG. 1, the system 100 may also include a data store 170 for storage of generated simulations.

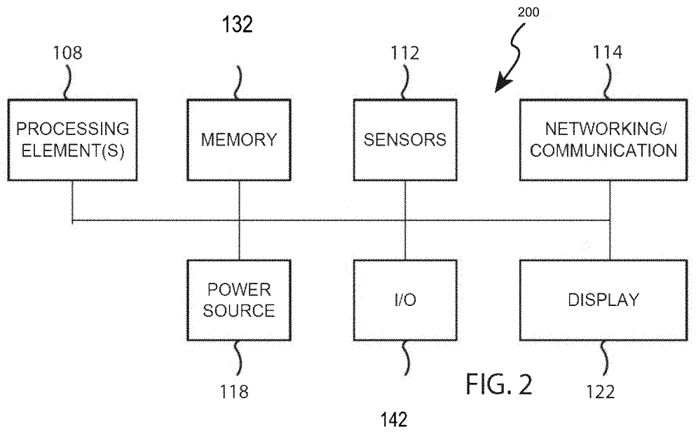

FIG. 2 is a simplified block diagram of a computing device. With reference to FIGS. 1 and 2, each of the elements of the SR risk mitigation system 100 may include one or more of the components shown in FIG. 2 such as one or more processing elements 108, one or more memory components 132, one or more sensors 112, a networking/communication interface 114, a sensor 116, a power source 118, an input/output interface 142, and/or a display 112. It should be noted that FIG. 2 is meant as exemplary only, in other examples the computing devices of the system 100 may include fewer or more components than those shown in FIG. 2.

The one or more processing elements 108 may be substantially any electronic device capable of processing, receiving, and/or transmitting instructions. For example, the processing element 108 may be a microprocessor or a microcomputer. Additionally, it should be noted that the processing element 108 may include more than one processing member. For example, a first processing element may control a first set of components of the computing device and a second processing element may control a second set of components of the computing device where the first and second processing elements may or may not be in communication with each other. Additionally, each processing element 108 may be configured to execute one or more instructions in parallel.

The memory 132 stores electronic data that may be utilized by the various elements of the risk mitigation system 100. For example, the memory 132 may store electrical data or content e.g. audio files, video files, document files, graphical interfaces, and so on, corresponding to various applications. The memory 132 may be, for example, non-volatile storage, a magnetic storage medium, optical storage medium, magneto-optical storage medium, read only memory, random access memory, erasable programmable memory, flash memory, or a combination of one or more types of memory components.

The sensors 112 may provide substantially any type of input to the system 100. For example, the sensors 112 may be one or more accelerometers, microphones, global positioning sensors, gyroscopes, light sensors, image sensors (such as a camera), force sensors, and so on. While any of the subsystems of the system 100 can include the sensors, in one embodiment the SR device 160 includes one or more of these sensors. The type, number, and location of the sensors 112 may be varied as desired and may depend on the desired functions of the system 100. In one example, the sensors 112 are location sensors that provide location information, such as GPS data, for the computing devices. The accuracy, format, and preciseness of the location may vary based on the type of SR device.

The networking/communication interface 114 receives and transmits data to and from the network 106 to each of the computing subsystems of system 100. The networking/communication interface 114 may transmit and send data to the network 106 and/or other computing devices. For example, the networking/communication interface may transmit data to and from other computing devices through the network 106, which may be a cellular or other wireless network (Wi-Fi, Bluetooth) or a wired network (Ethernet), or a combination thereof. The computing devices may also include a power supply 118. The power supply 118 provides power to various components of the computing devices. The power supply 118 may include one or more rechargeable, disposable, or hardwire sources, e.g. batteries, power cord, or the like. Additionally, the power supply 118 may include one or more types of connectors or components that provide different types of power to the computing devices.

The input/output interface 142 allows the computing devices to receive inputs from a user and provide output to the user. For example, the input/output interface 142 may include a capacitive touch screen, keyboard, mouse, stylus, or the like. The type of devices that interact via the input/output interface 120 may be varied as desired.

The display 122 provides a visual output for the computing devices. The display 122 may be substantially any size and may be positioned substantially anywhere on the computing devices. In some embodiments, the display 122 may be a liquid crystal display screen, plasma screen, light emitting diode screen, and so on. In some embodiments, the display 122 may also function as an input device in addition to displaying output from the computing device. For example, the display 122 may include capacitive touch sensors, infrared touch sensors, or the like that may capture a user's input to the display 122. In these embodiments, a user may press on the display 122 in order to provide input to the computer device. In other embodiments, the display 122 may be separate from or otherwise external to the electronic device, but may be in communication therewith to provide a visual output for the electronic device.

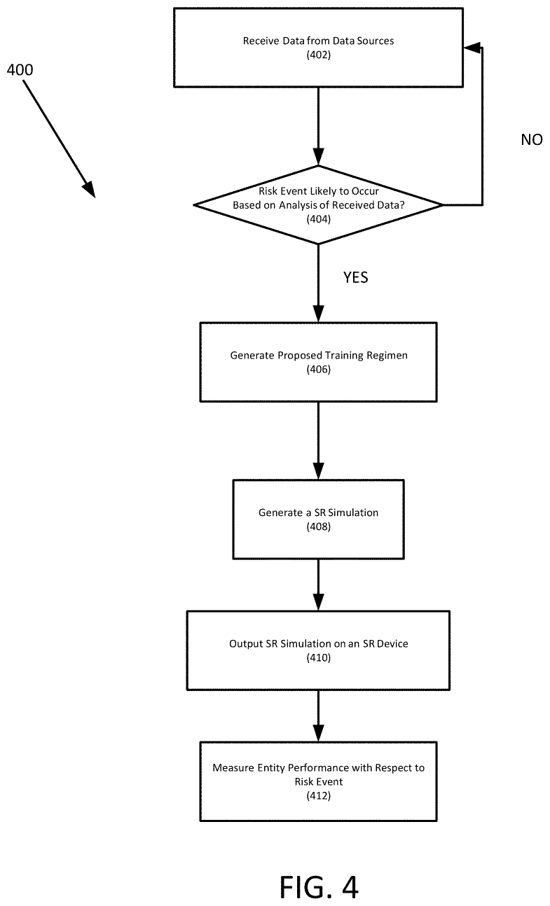

Referring now to FIG. 4, a flowchart illustrating an example method for identifying a risk event and generating a simulated training in the systems disclosed in FIG. 1 is shown. While the method 400 is described for the sake of convenience and not with an intent of limiting the disclosure as comprising a series and/or a number of steps, it is to be understood that the process does not need to be performed as a series of steps and/or the steps do not need to be performed in the order shown and described with respect to FIG. 4, but the process may be integrated and/or one or more steps may be performed together, or the steps may be performed in the order disclosed or in an alternate order.

At 402, the system may receive data from one or more sources. As discussed above, the data may internal data corresponding to an entity (e.g., financial records, HR records, etc.), and/or external data relating to the operations of an entity (e.g., compliance rules, news, etc.).

At 404, the system may analyze the received data to determine whether a risk event is going to occur or has a certain likelihood of occurrence (e.g., over a threshold). As discussed above, the system may make the determination using a predictive model and/or a rule set based on various risk factors (e.g., performance score, learning score, thresholds, target outcomes, etc.). Optionally, the risk event may be determined to occur or likely to occur if the current relative risk is over a threshold and/or generates a trigger. The system may continue performing steps 402-404 upon determining that risk event is not likely to occur (404: NO). However, if the risk event is determined to be likely to occur (404: YES), the system may generate a proposed training regimen (406) including one or more training modules, the training regimen designed to reduce the likelihood of occurrence to the risk event. Alternatively and/or additionally, the system may also generate an alert if the risk event is determined to be likely to occur.

The system may then generate a SR simulation (408) corresponding to each training module in the training regimens, as discussed above. For example, the system may identify components of the simulation such as assets, interactions, and/or environmental conditions and generate the simulation using the identified assets.

Optionally, the training regimen may be output to a user (e.g., via a client device) for approval before generation of the simulation. The user may approve, modify, or reject the training regimen. In some embodiments, the user's action with respect to the training regimen may be used as feedback for generation of future training regimens. For example, if a user modifies a training regimen to change its frequency from twice a year to 4 times a year for a particular risk event, the system may take this input into consideration for generation of other training regimens relating to that risk event and/or entity. Similarly, if a user modifies a training module simulation environment from an exam room to a medical ward, the system may use the medical ward for similar training simulations.

The system may then output the generated SR simulation (410) to a user (e.g., entity personnel) in accordance with the training regimen via an SR device. A user's performance may be measured during a simulated training, and appropriate feedback may be provided (e.g., a the system may notify regarding the mistakes made during training, provide material for improving performance, require the user to undergo another training simulation, repeat the training simulation, or the like).

Upon completion of a training module and/or a training regimen, the performance of the entity may be measured with respect to reduction in likelihood of the risk event (412). As discussed above, this performance data can be used to evaluate whether or not the simulation needs to be updated or if the simulation was suitable to mitigate the risk at issue. If the performance does not meet the threshold then the simulation is rejected and the system sends the information back to the training engine to propose a new simulation. This feedback loop allows for future improved performance of the system.

As discussed above, the systems and methods discussed above can be used in numerous ways, settings and entities, and are not limited to a medical facility setting. For example, the system may collect data relating to a commercial kitchen/food manufacturer from disparate sources and at different times. Example data sources may include radio-frequency identification (RFID) readings located on specific ingredients sourced (including food type, food source, dates associated with harvest, processing, spoilage, etc.), data related to the performance of the food manufacturer's production and processing equipment, data relating to shipments and deliveries, public statements and alerts from FDA and other regulatory agencies, or the like. The training engine 120 may find an elevated risk for food-borne illness based on temperature readings from cooking equipment, combined with changes in factory air quality and a poorly performing refrigeration unit on a delivery vehicle (either based on a predictive model using machine learning and/or using a rule set). Data sources informing the risk factor in this example may include a combination of social media postings, notifications from the consumer product safety commission, IoT alarms placed on the specific products in question. The system may verify that a specific alert threshold is met (e.g., number of potential affected consumers greater than a threshold), and can generate an alert to notify an administrator. As discussed above, the thresholds can be unique to each organization/user group, and can be set automatically by the training engine 120 based on predetermined performance targets, and/or manually adjusted. For example, a threshold may include a specific number of serious adverse events as they relate to baby and child-specific consumer product goods, such as cribs or basinets. For example, should the consumer product safety commission issue a product alert, yet no social media postings or IoT alarms have been triggers, the confluence of risk factors can be insufficient to the meet the threshold. However, should the consumer product safety commission issue a product alert, in conjunction with a rapid growth in the number of social media postings mentioning the product failure, the confluence of risk factors can be considered sufficient to meet the threshold. The training engine can alternatively and/or additionally determine the nature of the risk event (i.e., food-borne illness based on above risk factors) and prepare a proposed training regimen to mitigate the risk. For example, the training regimen may be determined based on a predictive model that has learned that keeping the cooking temperature above a certain temperature for a certain periods of time reduces the risk of food-borne illnesses for that particular entity (and/or a rule set defined based on best practices for cooking food in a commercial kitchen or manufacturing facility). The training regimen may, therefore, include a training module for employees involved in cooking within the entity that provide training about properly performing temperature checks and maintaining the required temperature for the required time. The system may then generate a simulated training by identifying assets such as cooking vessels, a stove, a temperature monitor, food ingredients (and their shape, color, texture, etc. at different temperature), or the like; and situating them in a proper environment (e.g., a commercial kitchen or cooking surface). The system may also identify and include the required interactions in the simulated training for performance measure (e.g., proper handling of temperature monitor for recording the temperature). The simulated training and/or the training regimen may be constantly updated based on new performance outcomes of the manufacturer.

In yet another example, the system may identify a risk event that a specific patient is at heightened risk (76%) for contracting a HA-CAUTI in the next 48 hours based on data received from, for example, health system data sources located across an entity's EMR, charting systems, laboratory results, ordering systems (labs, medication, etc.), staffing systems, etc. The identified risk factors may include: stroke patient unconscious and unresponsive (Source: EMR); admitted to the hospital intensive care unit (ICU) in the past 2 hours (Source: EMR); lab culture results indicate no infections upon admission (Source: Lab System and EMR); urinary catheter placed within the first hour of admission (Source: EMR & Ordering system); no antibiotics ordered by admitting or attending physician (Source: Ordering system); patient's attending ICU nurse has not completed routine urinary catheterization training since joining the health system in the past month (Source: Learning Management System); and nurse staffing shortage in the ICU (Source: Staff scheduling system). The risk factors may be used by the training engine (e.g., predictive model and/or rule set) to determine that the confluence of the above risk factors is indicative of the risk event. The threshold for generating an alert and/or training regimen may be heightened risk over 50% in the next 48 hours. The system may, therefore, generate a training regimen including, for example, training modules for training the nursing staff in proper placement of a urinary catheter. The system may identify the training regimen based on a predictive model (that has been trained to identify training s that will mitigate the risk event) and/or a rule set (determined based on, for example, best practices relating to the care of a stroke patient). The system may also generate an alerts for an administrator including information such as patient unique identifier; patient admission date and time; admitting and attending physicians and nurse manager; unit & location within health system; recommended trainees (clinical staff associated with the care of the patient(s) involved, such as nurses and physicians); a dataset order for recommended risk mitigation training modules based on the risk profile identified by the system viewable and editable through a web-based content-management system; a projection of the impact on risk over time (i.e. increases by 15% 24 hours from now) if left unaddressed; or the like.

By accepting the recommended risk mitigation training modules as is, the administrator informs the training engine that it has correctly identified the variables associated with the risk, correctly calculated the risk, and correctly recommended a training module tailored to mitigate the risk. If the administrator modifies the risk profile data based on user judgement, the training engine may determine that the predictive model has incorrectly identified the variables associated with the risk. The training engine receives the modifications made to the risk profile and adapts the predictive model accordingly. The training engine modifies the recommended risk-mitigation training module according to the revised risk profile. The training engine builds a simulated training accordingly. Upon rejection of training regimen, the training engine determines that the predictive model has incorrectly identified the variables associated with the risk. The training engine receives the rejection made to the risk profile and adapts the predictive model accordingly.

Upon acceptance of the training regimen by the administrator, the system may generate a training simulation relating to the training module. The simulation builder may the identify the specific components of the training simulation such as virtual 3D patient(s), virtual 3D environments, virtual medical history, virtual interactions (e.g., placement of a catheter on a virtual patient), competencies, etc. The server may output the simulated training module to the appropriate trainee's device (e.g., VR head mounted displays (HMD)). The system sends an SMS and/or email notification to the trainee that a new training module has been created for them based on the perceived organizational risk, and provides a time to live (TTL) certificate for completion. The trainee performs the training to demonstrate appropriate competency. The trainee's HMD tracks and reports performance data, informing the training engine of the trainees level of completion and competency. The training engine assesses clinical impact of the training module on risk-based outcomes (i.e. did the training mitigate the risk of HA-CAUTI) and adapts the predictive model accordingly.

The present disclosure is not to be limited in terms of the particular examples described in this application, which are intended as illustrations of various aspects. Many modifications and examples can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and examples are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions, or biological systems, which can, of course, vary.

While various aspects and examples have been disclosed herein, other aspects and examples will be apparent to those skilled in the art. The various aspects and examples disclosed herein are for purposes of illustration and are not intended to be limiting, with the true scope and spirit being indicated by the following claims.

What is claimed is:

1. A simulated reality (SR) generation system, comprising:
a processor; and
a non-transitory memory containing computer-readable instructions that when executed will cause the processor to:
receive information relating to an entity from one or more data sources;
analyze the received information to identify the need of a SR training simulation;
in response to the identification, generate the SR training simulation by identifying, based on the received information, at least one of the following SR simulation components:
a plurality of SR assets,
a plurality of interactions between a trainee and SR assets in the SR training simulation,
a plurality of interactions between SR assets in the SR training simulation, or
environmental conditions of the SR training simulation.

2. The SR generation system of claim 1, wherein identifying the need for the SR training simulation comprises:
determining a likelihood of occurrence of a risk event with respect to the entity; and
determining that the SR training simulation will reduce the likelihood of occurrence of the risk event.

3. The SR generation system of claim 1, further comprising programming instructions that when executed cause the processor to run the SR training simulation on a viewing device suitable for displaying display information in an SR environment based on the SR training simulation.

4. The SR generation system of claim 1, further comprising programming instructions that when executed cause the processor to:
receive a second set of information from the plurality of data source upon completion of the run of the SR training simulation;
analyze the received information to determine a performance measure of the entity; and
update the SR training simulation based on the analysis.

5. The SR generation system of claim 1, wherein identifying the environmental conditions of the SR training simulation comprises identifying the environmental conditions of the SR training simulation based on environmental conditions of a real-world scenario corresponding to the SR training simulation.

6. The SR generation system of claim 1, wherein identifying the plurality of interactions between a trainee and SR assets in the SR training simulation comprising identifying the plurality of interactions based on one or more of the following:
a risk factor for the entity associated with performance of the plurality of interactions;
trainee's performance record for performance of the plurality of interactions; or trainee's previous training record for performance the plurality of interactions.

7. The SR generation system of claim 1, wherein identifying the plurality of assets comprises identifying the assets based on:
a risk factor for the entity associated with an asset;
trainee's performance record associated with interactions with an asset;
trainee's previous training record associated with interactions with an asset; or
a real-world scenario associated with the SR training simulation.

8. The SR generation system of claim 1, wherein the plurality of assets comprise at least one of the following: objects, virtual beings, or an environment.

9. The SR generation system of claim 1, wherein the programming instructions that cause the processor to generate the SR training simulation comprise instructions that cause the processor to identify the SR simulation components comprising:
the plurality of SR assets; and
the plurality of interactions between the trainee and SR assets in the SR training simulation.

10. The SR generation system of claim 1, wherein the programming instructions that cause the processor to generate the SR training simulation comprise instructions that cause the processor to identify the SR simulation components comprising:
the plurality of SR assets;
the plurality of interactions between the trainee and SR assets in the SR training simulation; and
environmental conditions of the SR training simulation.

11. A risk mitigation simulated reality (SR) system, comprising:
a viewing device suitable for displaying display information in an SR environment based on a SR training simulation;
an input device configured to allow a user to effect that training simulation;
a processor; and
a non-transitory memory containing computer-readable instructions that when executed will cause the processor to:
receive information relating to an entity from a one or more data sources;
analyze the received information to identify a likelihood of occurrence of a risk event with respect to the entity;
generate a SR training simulation for reducing the likelihood of occurrence of the risk event, and
run the SR training simulation on the viewing device.

12. The risk mitigation SR system of claim 11, wherein analyzing the received information to identify the likelihood of occurrence of the risk event with respect to the entity comprises analyzing at least one of the following risk factors: a performance score of the entity determined based on past performance of the entity corresponding to the risk event; a learning score of the entity determined based on training history of the entity corresponding to the risk event; various thresholds corresponding to the risk event; a target outcome relating to the risk event; or real-time data associated with the risk event.

13. The risk mitigation SR system of claim 11, further comprising programming instructions that when executed cause the processor to determine whether the identified likelihood of occurrence of the risk event with respect to the entity corresponds to a trigger event.

14. The risk mitigation SR system of claim 13, further comprising programming instructions that when executed cause the processor to generate an alert on a user device in response to determining that the identified likelihood of occurrence of the risk event with respect to the entity corresponds to the trigger event.

15. The risk mitigation SR system of claim 11, further comprising programming instructions that when executed cause the processor to generate a training regimen based on an analysis of the risk event and the received information, the training regimen configured for reducing the likelihood of occurrence of the risk event.

16. The risk mitigation SR system of claim 15, wherein the training regimen comprises at least one of the following: one or more training modules, frequency of training, required performance measures, training criteria, or identification of trainees.

17. The risk mitigation SR system of claim 15, wherein the SR training simulation is generated to run a training module included in the training regimen in simulated reality.

18. The risk mitigation SR system of claim 11, further comprising programming instructions that when executed cause the processor to receive user approval of the training regimen before generation of the SR training simulation.

19. The risk mitigation SR system of claim 11, wherein generating the SR training simulation comprises identifying, based on a training module, at least one of the following:
a plurality of SR assets;
a plurality of interactions between a trainee and SR assets in the SR training simulation;
a plurality of interactions between SR assets in the SR training simulation;
environmental conditions of the SR training simulation; or
one or more performance metrics of a user participating in the SR training simulation.

20. The risk mitigation SR system of claim 11, further comprising programming instructions that when executed cause the processor to:
receive a second set of information from the plurality of data source upon completion of the run of the SR training simulation;
analyze the received information to determine a performance measure of the entity with respect to the risk event; and
update the SR training simulation based on the analysis.
